(19) United States
(12) Patent Application Publication
(10) Pub. No.: US 2025/0182230 A1
(43) Pub. Date: Jun. 5, 2025

(54) GUIDANCE ASSISTANCE APPARATUS, GUIDANCE ASSISTANCE METHOD, AND COMPUTER-READABLE RECORDING MEDIUM

(71) Applicant: NEC Corporation, Minato-ku, Tokyo (JP)

(72) Inventors: Shuntaro Jimba, Tokyo (JP); Yasuyuki Chikushi, Tokyo (JP); Tatsuya Ishikawa, Tokyo (JP)

(73) Assignee: NEC Corporation, Minato-ku, Tokyo (JP)

(21) Appl. No.: 18/833,082

(22) PCT Filed: Mar. 18, 2022
(86) PCT No.: PCT/JP2022/012695

§ 371 Date: Feb. 21, 2025

(51) Int. Cl. G06Q 50/20; A61B 5/16; G16H 20/70; G16H 40/67

(52) U.S. Cl. CPC G06Q 50/205; G16H 20/70; G16H 40/67; A61B 5/165; A61B 2503/12

(57) ABSTRACT

There is provided a guidance assistance apparatus 100 including: an acquisition unit 110 acquires biological information of a student measured using a wearable terminal 300 worn by the student S; an estimation unit 120 estimates a feeling of the student S based on the acquired biological information; and a presentation unit 130 presents condition information including the feeling of the student S, together with extracurricular activity information on the student's extracurricular activity, on a user terminal used by a teacher.

### TECHNICAL FIELD

The present disclosure relates to a guidance assistance apparatus, a guidance assistance method, and a computer-readable recording medium.

### BACKGROUND ART

In the field of school education, schools (teachers) are required to provide guidance tailored to each student so that they can support students in balancing schoolwork and extracurricular activities. Communication between teachers and students is important in order to provide guidance tailored to each student. However, as remote classes increase, opportunities for teachers and students to meet face-to-face will decrease, making it difficult for the teachers to grasp the status of their students.

As a technology to assist teachers in providing guidance to students, for example, Patent Document 1 discloses a technology for determining a weight value for each student from an index value related to the student's learning behavior, correcting the results of survey on each student's class satisfaction based on the weight value, and presenting the corrected results to the teacher. In addition, Patent Document 2 discloses a learning support system that obtains a student's response to information transmitted to the student, estimates the student's current feelings based on the obtained response, determines content of information to be transmitted to the student based on the estimated feelings, and transmits the information.

### LIST OF RELATED ART DOCUMENTS

### Patent Document

Patent Document 1: JP2014-238649A
Patent Document 2: JP2021-108046A

### SUMMARY OF INVENTION

### Problems to be Solved by the Invention

However, in the technologies described in Patent Documents 1 and 2, the status of students' extracurricular activities are not considered. In order for teachers to provide guidance tailored to each student while taking into account balancing schoolwork and extracurricular activities, it is necessary to provide the teachers with information considering not only the status of the student's schoolwork but also the status of the student's extracurricular activities.

An example object of the present disclosure is to provide a guidance assistance apparatus, a guidance assistance method, and a computer-readable recording medium with which teachers can be provided with information useful for giving guidance tailored to each student with consideration of balancing between schoolwork and extracurricular activities.

### Means for Solving the Problems

In order to achieve the above object, a guidance assistance apparatus includes:

an acquisition means for acquiring biological information of a student measured using a wearable terminal worn by the student:

an estimation means for estimating a feeling of the student based on the acquired biological information; and

a presentation means for presenting condition information including the feeling of the student, together with extracurricular activity information on the student's extracurricular activity, on a user terminal used by a teacher.

Also, in order to achieve the above object, a guidance assistance method includes:

acquiring biological information of a student using a wearable terminal worn by the student:

estimating a feeling of the student based on the acquired biological information; and

presenting condition information including the feeling of the student to a user terminal used by a teacher, together with extracurricular activity information on the student's extracurricular activity.

Furthermore, in order to achieve the above object, a computer readable recording medium that includes a program recorded thereon, the program including instructions that causes a computer to function as a guidance assistance apparatus, wherein

the guidance assistance apparatus includes:

an acquisition means for acquiring biological information of a student measured using a wearable terminal worn by the student:

an estimation means for estimating a feeling of the student based on the acquired biological information; and

a presentation means for presenting condition information including the feeling of the student, together with extracurricular activity information on the student's extracurricular activity, on a user terminal used by a teacher.

### Advantageous Effects of the Invention

As described above, according to the present invention, it can be provided with information useful for giving guidance tailored to each student with consideration of balancing between schoolwork and extracurricular activities.

### BRIEF DESCRIPTION OF THE DRAWINGS

FIG. 1 is a block diagram illustrating a configuration example of a guidance assistance apparatus according to an example embodiment.

FIG. 2 is an explanatory diagram illustrating a configuration example of a guidance assistance system that includes a guidance assistance apparatus according to an example embodiment.

FIG. 3 is an explanatory diagram illustrating an example of student condition information.

FIG. 4 is an explanatory diagram illustrating an example of student basic information.

FIG. 5 is a timing chart illustrating an example of a guidance assistance method according to an example embodiment.

FIG. 6 is a list screen displayed on a display unit of a user terminal, illustrating a list of students who take a certain class.

FIG. 7 is an explanatory diagram illustrating an example of display a pop-up screen on a list screen.

FIG. 8 is an explanatory diagram illustrating an example of a pop-up screen.

FIG. 9 is an explanatory diagram illustrating an example of a questionnaire screen displayed on the student's terminal.

FIG. 10 is a block diagram illustrating a configuration example of a guidance assistance apparatus is constituted by a user-side apparatus and a server-side apparatus.

FIG. 11 is a block diagram illustrating one example of a computer that realizes a guidance assistance apparatus according to an example embodiment.

### EXAMPLE EMBODIMENTS

### 1. Overview

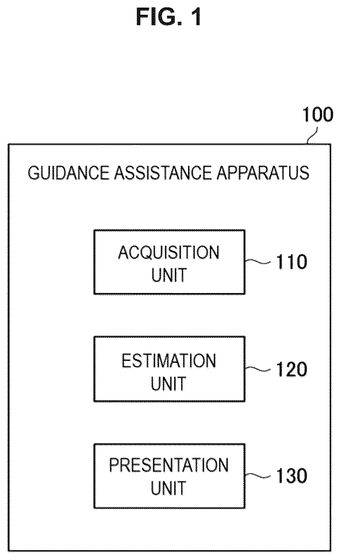

First, a configuration and functions of a guidance assistance apparatus 100 according to an example embodiment will be described with reference to FIG. 1. FIG. 1 is a block diagram showing a configuration example of the guidance assistance apparatus 100 according to the example embodiment.

The guidance assistance apparatus 100 is an apparatus that provides teachers with information that is useful for providing guidance tailored to each student with consideration of balancing between schoolwork and extracurricular activities. The guidance assistance apparatus 100 includes an acquisition unit 110, an estimation unit 120, and a presentation unit 130, as shown in FIG. 1.

The acquisition unit 110 acquires each student's biological information. The student's biological information can be measured by a wearable terminal worn by the student. The estimation unit 120 estimates the student's feelings based on the biological information acquired by the acquisition unit 110. The presentation unit 130 presents condition information including the student's feelings estimated by the estimation unit 120 to a user terminal used by the teacher, along with extracurricular activity information on the student's extracurricular activities.

The guidance assistance apparatus 100 allows the teacher to grasp the latest condition information indicating how students are feeling, along with information on their extracurricular activities. This makes it possible to assist teachers in providing individualized guidance tailored to each student while reducing their workload.

### 2. Apparatus Configuration

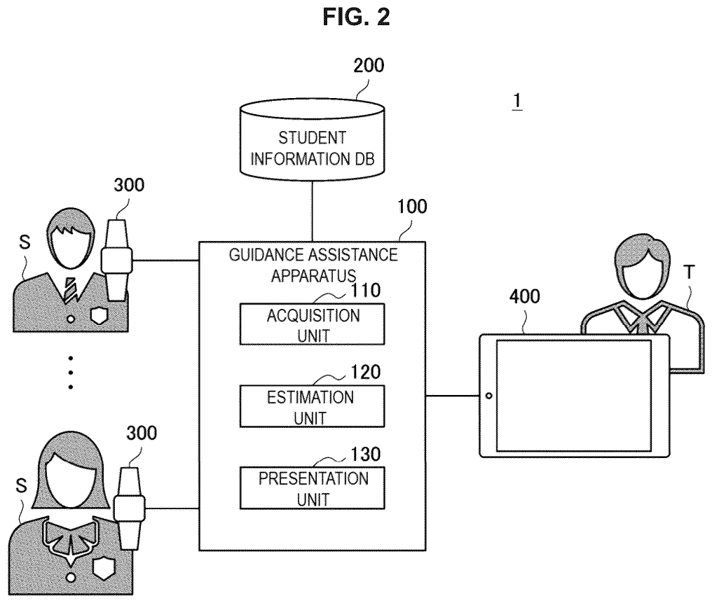

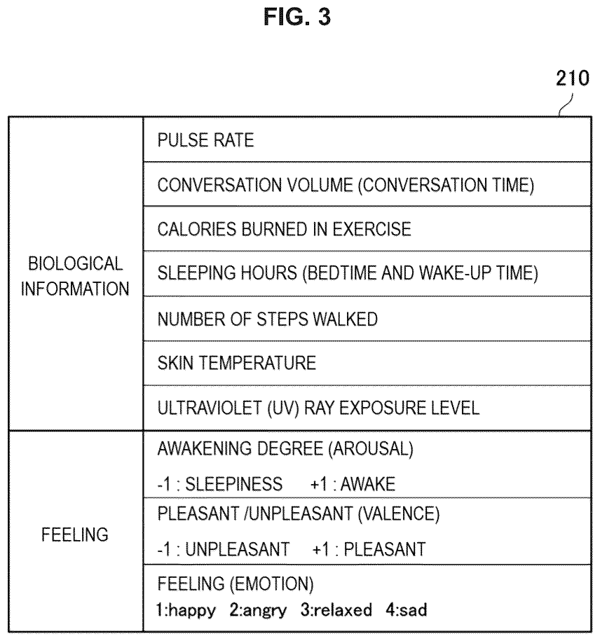

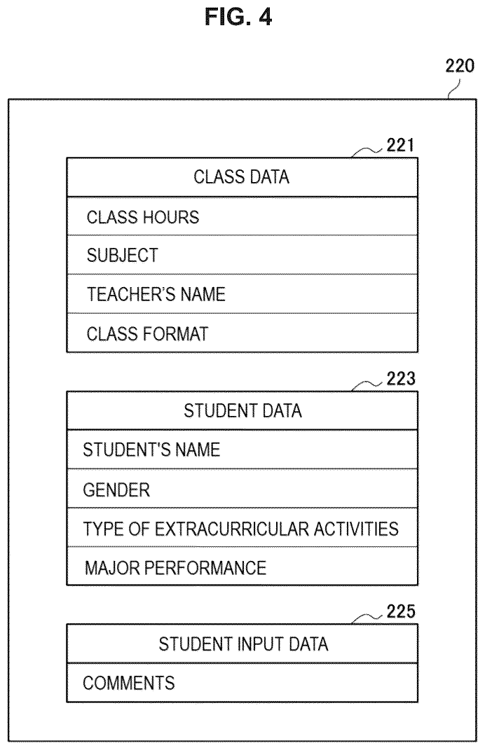

A configuration and functions of the guidance assistance apparatus 100 according to the example embodiment will be described in more detail with reference to FIGS. 2 to 4. FIG. 2 is an explanatory diagram showing a configuration example of a guidance assistance system 1 that includes the guidance assistance apparatus 100 according to the example embodiment. FIG. 3 is an explanatory diagram showing an example of student condition information. FIG. 4 is an explanatory diagram showing an example of student basic information.

As shown in FIG. 2, in the guidance assistance system 1, the guidance assistance apparatus 100, a student information database (DB) 200 that stores student condition information and basic information, wearable terminals 300 worn by students S, and a user terminal 400 used by a teacher T are communicably connected via a network.

Each student S wears the wearable terminal 300 that measures biological information. The wearable terminal 300 is a computer device that is put on the wrist, arm, head, or the like of a user (student) whose biological information is to be measured. Although the form of the wearable terminal 300 is not particularly limited, the wearable terminal 300 may be of a wristwatch type, wristband type, or the like, for example. The wearable terminal 300 is associated with the student S. For example, associating the identification information of the wearable terminal 300 (terminal ID) with the identification information of the student (student ID) makes it possible to identify the student to which the biological information measured by the wearable terminal 300 belongs.

The wearable terminal 300 measures the student's biological information such as pulse rate, sleeping hours, number of steps walked, and skin temperature. Examples of the biological information measured by the wearable terminal 300 includes pulse rate, conversation volume (conversation time), calories burned in exercise, sleeping hours (bedtime and wake-up time), number of steps walked, skin temperature (body temperature), ultraviolet (UV) ray exposure level, and the like. The wearable terminal 300 transmits the measured biological information to the guidance assistance apparatus 100 via the network. The guidance assistance apparatus 100 records the received biological information in the student information DB 200 in association with the identification information (student ID) for each student.

The guidance assistance apparatus 100 estimates the student's feelings based on the student's biological information, and presents condition information including the estimated student's feelings to the user terminal 400 used by the teacher, along with extracurricular activity information on the student's extracurricular activities. The guidance assistance apparatus 100 includes the acquisition unit 110, the estimation unit 120, and the presentation unit 130, as shown in FIG. 1. The acquisition unit 110 acquires student's biological information stored in the student information DB 200 and transmits the same to the estimation unit 120.

The estimation unit 120 estimates each student's feeling based on the biological information acquired by the acquisition unit 110. The estimation unit 120 estimates, from the acquired biological information, how the student was feeling at the time when the biological information was acquired. The estimation unit 120 estimates, as the feeling, at least one of arousal, valence, and emotion, for example.

Arousal is generally an index that indicates the strength and height of aroused emotions, and shows the physiological activity state of the brain. For example, it can be an index that indicates whether the target person is in a sleepy state. In the present example embodiment, the arousal (awakening degree) is an index that expresses whether the target person is in a sleepy state in a binary representation or in a stepwise manner, with a state with sleepiness being “−1” and an awake state without sleepiness being “+1”. The arousal may be estimated using a known technology (for example, International Publication No. 2018/179289, International Publication No. 2018/179292, International Publication No. 2019/146123, International Publication No. 2020/157989, or the like). For example, the estimation unit 120 may regard the pulse rate as the heart rate and determine the arousal based on a value equivalent to the average value of RR intervals (average RR interval).

Valence is an index that indicates the direction of emotion evoked, and can be used as an index that indicates whether the feeling is positive, for example. In the present example embodiment, the valence is an index that expresses the direction of emotion in a binary representation or in a stepwise manner, with an unpleasant (negative) state being “−1” and a pleasant (positive) state being “+1”. The valence may also be estimated using a known technology (for example, Japanese Patent Laid-Open Publication No. 2019-063324, Japanese Patent Laid-Open Publication No. 2021-157609A, or the like). For example, the estimation unit 120 may regard the pulse rate as the heart rate and determine the valence based on a value equivalent to the standard deviation of the amplitude of a low frequency component of the RR interval (low frequency (LF) amplitude standard deviation).

Emotion is the index of feelings, which represents a group of feelings such as “happy.” “relaxed,” “angry,” and “sad.” for example. In the present example embodiment, the emotion is the index that indicates four feelings, with happy as “1”, relaxed as “2”, angry as “3”, and sad as “4”. The emotion may also be estimated using a known technology (for example, Japanese Patent Laid-Open Publication No. 2019-063324, International Publication No. 2015/182077, International Publication No. 2019/069910, International Publication No. 2020/157988, or the like). For example, the estimation unit 120 may regard the pulse rate as the heart rate and determine the emotion based on a value equivalent to the average RR interval and the LF amplitude standard deviation.

The estimation unit 120 may also estimate feelings other than arousal, valence, and emotion. The estimation unit 120 may further estimate information on the feelings based on any of information on the estimated arousal, valence, and emotion. The estimation unit 120 records the estimated feelings in the student information DB 200 in association with the identification information (student ID) for each student.

The presentation unit 130 presents the condition information including the student's feelings estimated by the estimation unit 120 to the user terminal 400 used by the teacher, along with extracurricular activity information on the student's extracurricular activities. The presentation unit 130 presents, as the student condition information, the feelings estimated by the estimation unit 120 and various kinds of biological information measured by the wearable terminal 300 worn by the student to the user terminal 400. The presentation unit 130 also presents the student's extracurricular activity type, performance in the extracurricular activities, and the like to the user terminal 400, as the extracurricular activity information on the student's extracurricular activities.

The user terminal 400 is an information processing apparatus used by the teacher T, which is a tablet terminal, a personal computer, or the like, for example. The teacher T grasps the student condition information and extracurricular activity information through the display unit of the user terminal 400).

As described above, presenting the student condition information and on extracurricular activity information from the presentation unit 130 to the user terminal 400 allows the teacher T using the user terminal 400 to grasp the student S's feelings and activity status of extracurricular activities. Accordingly, it is possible to assist the teacher T in providing individualized guidance tailored to each student, with consideration of not only his/her study status but also hir/her extracurricular activity status. A method for presenting the information to the user terminal 400 by the presentation unit 130 will be described later.

Various kinds of information on students is stored in the student information DB 200. The student information DB 200 stores student condition information 210 as shown in FIG. 3, for example. In the student information DB 200, the condition information 210 is recorded in association with identification information (student ID) for each student. The condition information 210 includes the student's biological information measured by the wearable terminal 300 and the feelings estimated by the estimation unit 120 of the guidance assistance apparatus 100. The biological information and feelings are recorded as time series data in association with the measurement time of the biological information. The condition information 210 may be stored in the student information DB 200 at the timing when the guidance assistance apparatus 100 acquires biological information or estimates feelings, for example.

The student information DB 200 also stores student basic information 220 as shown in FIG. 4, for example. The basic information includes data on classes taken by the student (class data) 221, personal data on the student (student data) 223, data input by the student (student input data) 225, and the like, for example.

The class data 221 is information generated for each class taken by the student, and includes class hours, subject, teacher's name, and class format (for example, face-to-face class, online class, or the like), for example. The student data 223 includes information for identifying each individual student such as the student's name and gender, and information on extracurricular activities such as the type of extracurricular activities and major performance in the extracurricular activities, for example. The student input data 225 includes comments or the like to the teacher input by the student from his/her own terminal, for example. The student input data 225 may be additionally recorded in the student information DB 200. The basic information can be updated at any time. A configuration may be also be adopted in which, when new information is input by the student or the teacher, the new information is reflected on the information regarding extracurricular activities in the student data 223, the student input data 225, and the like, at the time of input.

### 3. Guidance Assistance Method

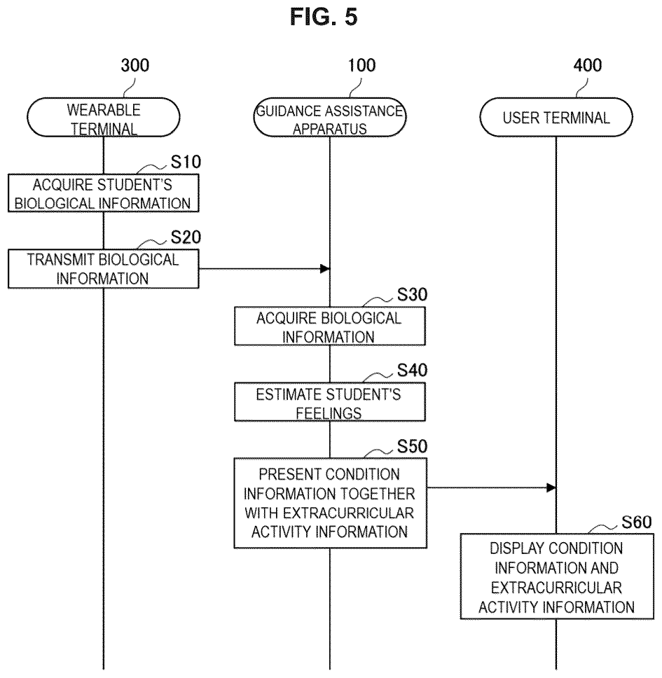

Hereinafter, a guidance assistance method according to an example embodiment will be described with reference to FIG. 5. FIG. 5 is a timing chart showing an example of the guidance assistance method according to the example embodiment. The guidance assistance method according to the example embodiment is executed by operating the guidance assistance apparatus 100.

### (S10 and S20: Acquisition of Biological Information)

In the guidance assistance method according to the example embodiment, as shown in FIG. 4, first, the wearable terminal 300 worn by a student acquires student's biological information (S10), and transmits the acquired biological information to the guidance assistance apparatus 100 at a predetermined timing (S20). The student basically wears the wearable terminal 300 at all times, and various kinds of biological information is measured as data on daily activities. The wearable terminal 300 transmits the measured biological information to the guidance assistance apparatus 100 at a predetermined timing (for example, every hour).

### (S30 and S40: Estimation of Student's Feelings)

The guidance assistance apparatus 100 acquires the student's biological information using the acquisition unit 110 from the student information DB 200 at a predetermined timing (S30), and estimates the student's feelings by the estimation unit 120 (S40). The acquisition unit 110 acquires the student's biological information from the student information DB 200 at a predetermined timing, for example, every half day or every day: Then, the estimation unit 120 estimates the student's feelings based on the student's biological information acquired by the acquisition unit 110. As described above, the estimation unit 120 estimates the arousal, valence, emotion, and the like as the feelings, for example. The estimation unit 120 stores the estimated feelings in the student information DB 200.

The estimation unit 120 may further estimate the student's feelings in a manner separated into feelings toward schoolwork and feelings toward extracurricular activities. For example, the feelings toward extracurricular activities are estimated based on biological information measured during the time period when the student is participating in extracurricular activities (for example, early morning exercise time, after-school time, or the like). The time period for extracurricular activities may be recorded in the student data 223 in advance. On the other hand, the feelings toward schoolwork are estimated based on biological information measured during time periods other than the time period of extracurricular activities.

Alternatively, if the time period of extracurricular activities and other time periods can be set on the wearable terminal 300, the student may be enabled to operate the wearable terminal 300) to distinguish whether the measured biological information is from the time period set for the extracurricular activities or the other time period. In this case, the wearable terminal 300 transmits information for distinguishing the acquisition time period to the guidance assistance apparatus 100 along with the measured biological information.

The estimation unit 120 may use different techniques to estimate the feelings toward schoolwork and the feelings toward extracurricular activities. For example, when estimating the feelings toward schoolwork, the estimation unit 120 may take into consideration the student's grades, class attendance status, assignment submission status, and the like. Further, when estimating feelings toward extracurricular activities, the estimation unit 120 may take into consideration performance in the extracurricular activities, participation status in the extracurricular activities, and the like, for example.

### (S50 and S60: Presentation of Condition Information and Extracurricular Activity Information)

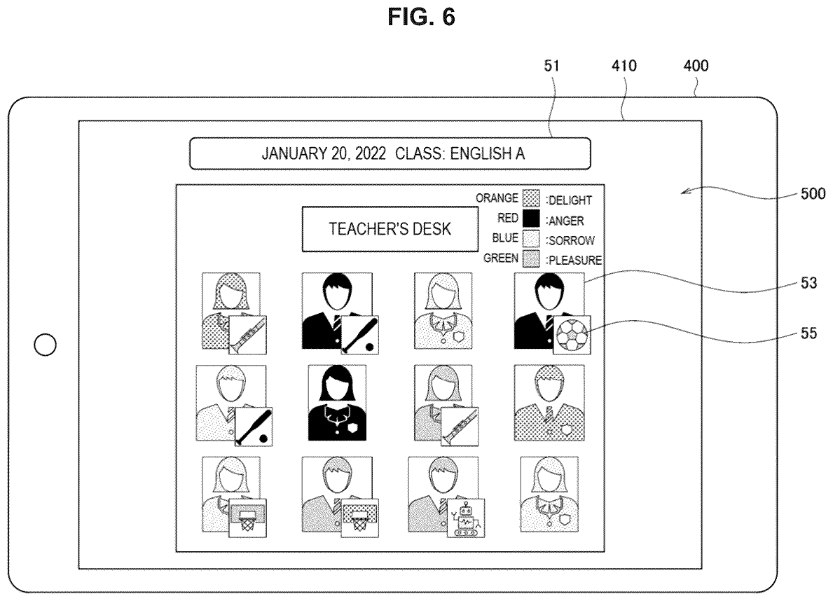

After that, the presentation unit 130 presents the condition information including the student's feelings together with the extracurricular activity information to the user terminal 400 used by the teacher (S50). Based on the information from the presentation unit 130, the condition information and extracurricular activity information are displayed on the display unit of the user terminal 400 (S60). FIGS. 6 to 8 show presentation examples of condition information and extracurricular activity information on the user terminal 400.

FIG. 6 is a list screen 500 displayed on a display unit 410 of the user terminal 400, showing a list of students who take a certain class. On the list screen 500, the class date and time and the class name are presented as class information 51. Further, student image objects 53 representing students and extracurricular activity objects 55 representing extracurricular activity information of the students are associated and presented on the list screen 500, in correspondence with the seat positions in the actual classroom.

The student image objects 53 are images representing the students, and may be thumbnails of students' photos or illustrations. Alternatively, the student image objects 53 may be displayed together with the students' names. In this case, all the images of the student image objects 53 may be the same. The extracurricular activity objects 55 are presented together with the student image objects 53 for students who participate in the extracurricular activities. The types of extracurricular activities may be represented by marks as shown in FIG. 6. For example, the state of belonging to a soccer club is represented by a soccer ball mark, the state of belonging to a baseball club is represented by a bat-and-baseball mark, and the state of belonging to a brass band club is represented by a musical instrument mark.

The student image objects 53 and the extracurricular activity objects 55 are presented on the list screen 500 in a distinguishable manner so that students' feelings can be understood. For example, in the example shown in FIG. 6, feelings are represented by four colors, orange, red, blue, and green (the colors are expressed by hatching in FIG. 6), Orange represents “delight”, red represents “anger”, blue represents “sorrow”, and green represents “pleasure”, In this example, the extracurricular activity objects 55 represent feelings toward extracurricular activities, and the student image objects 53 represent feelings toward schoolwork (other than extracurricular activities). The feelings toward extracurricular activities and the feelings toward schoolwork are estimated by the estimation unit 120. In this way, since the feelings toward extracurricular activities and the feelings toward schoolwork are represented by changing colors, teachers can easily grasp whether the students are enjoying their classes and extracurricular activities.

In the example of FIG. 6, the students' feelings are expressed by different-colored objects, but they may be displayed using other presentation methods. For example, the student image objects 53 and the extracurricular activity objects 55 may each be represented in a pie chart that shows the percentages of “delight”. “anger”. “sorrow”, and “pleasure”. The list screen 500 shown in FIG. 6 can also be presented on the user terminal 400 in real time during class.

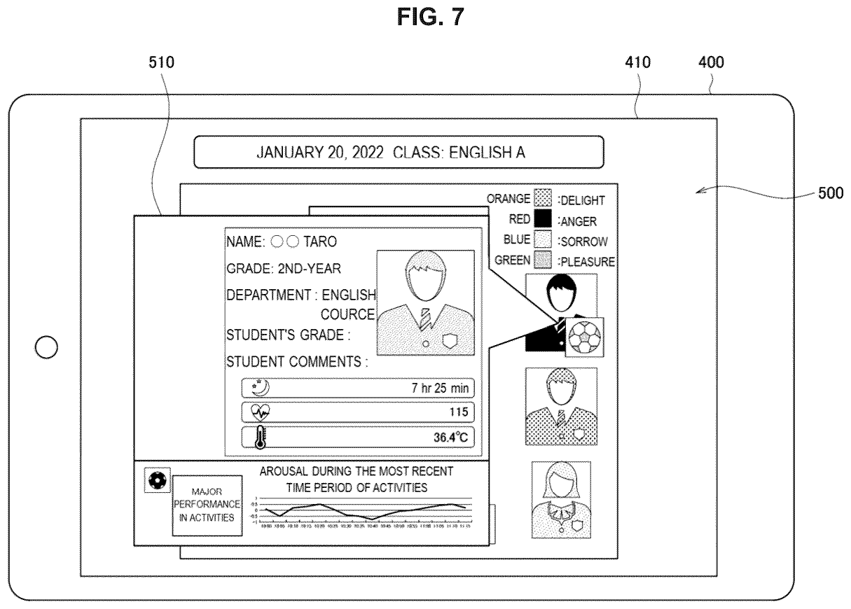

When the teacher selects the student image object 53 of one student from the list screen 500 using the user terminal 400, the presentation unit 130 may display detailed information of the selected student on a pop-up screen 510, as shown in FIG. 7.

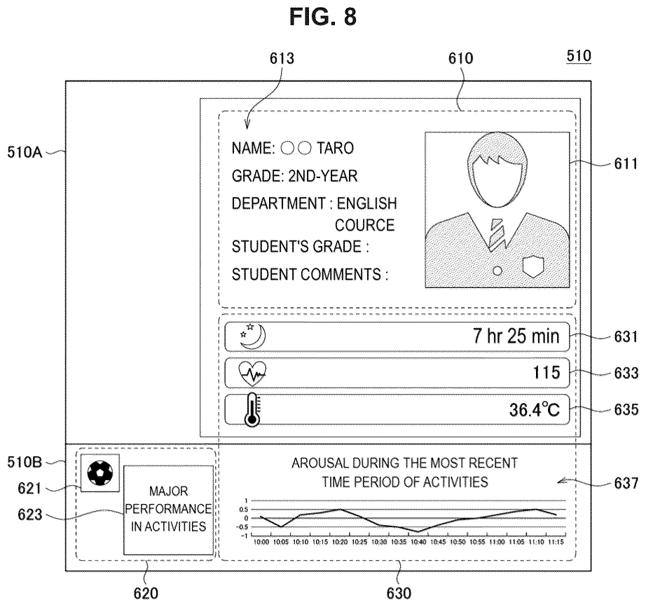

FIG. 8 shows an example of the pop-up screen 510. The pop-up screen 510 includes a schoolwork information area 510A that shows information on schoolwork, and an extracurricular activity information area 510B that shows information on extracurricular activities, for example.

Presented in the schoolwork information area 510A are a student image 611 and basic information 613 such as name, grade, department, and the like, for example. The student image 611 may be the same as the student image object 53 displayed on the list screen 500 in FIG. 6, or may be a photo of the student. In addition, the student's grade, comments input by the student (student comments), and the like may be presented in the schoolwork information area 510A. These are basic information on the student and are stored as student data 223 in the student information DB 200. Furthermore, in the schoolwork information area 510A, sleeping hours 631, heart rate (pulse rate) 633, body temperature (skin temperature) 635, and the like may be displayed as student condition information 630.

Presented in the extracurricular activity information area 510B are an extracurricular activity type 621, extracurricular activity performance information 623, and extracurricular activity feeling information 637, for example. The extracurricular activity type 621 is information for specifying extracurricular activities, and may be the same as the extracurricular activity object 55 displayed on the list screen 500 in FIG. 6, or may be text information. The extracurricular activity type 621 and the extracurricular activity performance information 623 are student basic information, and are stored as the student data 223 in the student information DB 200. The extracurricular activity feeling information 637 is one piece of the student condition information 630, and presents at least one of the feelings estimated by the estimation unit 120. For example, in FIG. 8, the arousal during the most recent time period of extracurricular activities is shown as time series data. The arousal can be calculated based on the performance of extracurricular activities and the biological information such as pulse rate, for example.

In this way, presenting the student's detailed information on the pop-up screen 510 allows the teacher to grasp the student's detailed status in schoolwork and extracurricular activities.

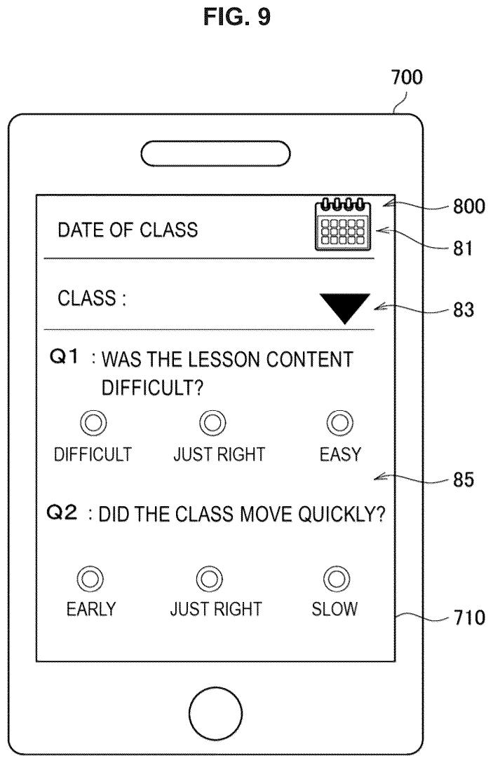

The guidance assistance apparatus 100 may further be prepared to obtain the students impressions on a class such as the progress and difficulty of the class, by questionnaires. The responses to the questionnaires serve as an indicator of each student's degree of understanding of the class. FIG. 9 shows an example of a questionnaire screen 800 displayed on the student's terminal 700. The student's terminal 700 may be the same as the wearable terminal 300, or may be another information processing terminal such as a tablet terminal or a personal computer.

The questionnaire screen 800 is displayed on a display unit 710 of the terminal 700. The questionnaire screen 800 includes a class date selection area 81, a class selection area 83, and a question area 85, for example. The student selects the date of a class about which the questionnaire will be conducted in the class date selection area 81, for example. The class date can be input by selecting a calendar object and selecting a date on the calendar. The student also selects the class about which the questionnaire will be conducted in the class selection area 83. Selectable classes are displayed in a pull-down list. The student selects from the list the class about which he/she will respond to the questionnaire. After selecting the class date and class, the student makes responses to the questions displayed in the question area 85. The responses to the questionnaire are sent from the student's terminal 700 to the guidance assistance apparatus 100.

The questionnaire responses are stored in the student information DB 200, for example. When the presentation unit 130 displays the pop-up screen 510 on the teacher's user terminal 400, the guidance assistance apparatus 100 may display the questionnaire responses on the pop-up screen 510, for example. This allows the teacher to grasp the student's degree of understanding of the class.

The guidance assistance method according to one example embodiment has been described above. In the guidance assistance method according to the example embodiment, the condition information including students' feelings estimated from the students' biological information is presented to the user terminal used by the teacher, along with the extracurricular activity information on the students' extracurricular activities. This allows the teacher to accurately grasp the latest information on the students' schoolwork and extracurricular activities. This makes it possible to assist the teacher in providing individualized guidance tailored to each student while reducing his/her workload. Furthermore, since it is possible to grasp trends in the relationship between each student's degree of enrichment in non-schoolwork activities such as extracurricular activities and schoolwork performance, the teacher can use this information to determine the guidance policy.

### 4. Program

A program according to the example embodiment is a program for causing a computer to function as the functional units shown in FIG. 1. Installing and executing this program on the computer implements the guidance assistance apparatus 100 and the guidance assistance method according to the example embodiment. In this case, the processor of the computer functions as the acquisition unit 110, the estimation unit 120, and the presentation unit 130 of the guidance assistance apparatus 100, and performs processing.

In one example embodiment, the student information DB 200 may be implemented by storing data files constituting the student information DB 200 in a storage device such as a hard disk included in the computer, or may be realized by a storage device of another computer. The computers may be general-purpose PCs, smartphones, or tablet terminal devices.

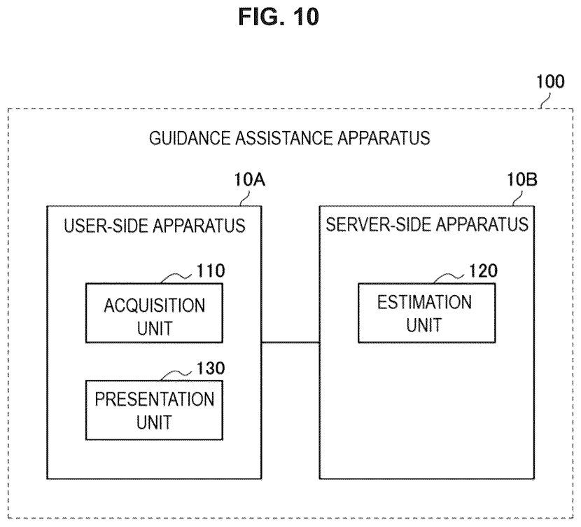

The program according to the example embodiment may further be executed by a computer system constructed by a plurality of computers. In this case, each computer may function as the acquisition unit 110, the estimation unit 120, or the presentation unit 130. For example, as shown in FIG. 10, the guidance assistance apparatus 100 may be constituted by a system that includes a user-side apparatus 10A having the acquisition unit 110 and the presentation unit 130, and a server-side apparatus 10B having the estimation unit 120.

In the system constituting the guidance assistance apparatus 100 shown in FIG. 10, the user-side apparatus 10A uses the acquisition unit 110 to acquire the student's biological information measured by the wearable terminal 300 worn by the student. The user-side apparatus 10A also uses the presentation unit 130 to present the condition information including the student's feelings estimated by the server-side apparatus 10B to the user terminal 400, along with information on the student's extracurricular activities. On the other hand, the server-side apparatus 10B estimates the student's condition including feelings based on the biological information acquired by the user-side apparatus 10A, and transmits the estimated condition to the user-side apparatus 10A.

### 5. Physical Configuration

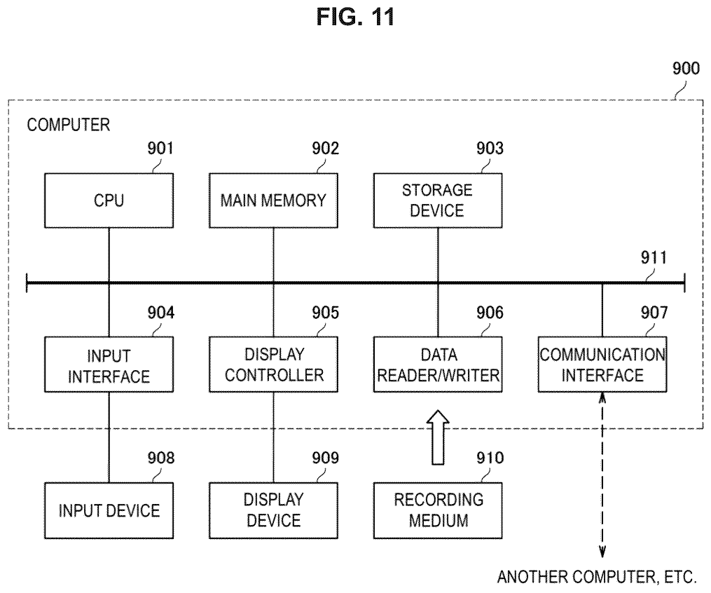

Using FIG. 11, a description is now given of a computer that realizes the guidance assistance apparatus 100 by executing the program according to the present example embodiment. FIG. 11 is a block diagram illustrating one example of the computer 900 that realizes the guidance assistance apparatus 100 according to the example embodiment.

As illustrated in FIG. 11, a computer 900 includes a CPU (Central Processing Unit) 901, a main memory 902, a storage device 903, an input interface 904, a display controller 905, a data reader/writer 906, and a communication interface 907. These components are connected in such a manner that they can perform data communication with one another via a bus 911.

Also, a computer 900 may include a GPU (Graphics Processing Unit) or an FPGA (Field-Programmable Gate Array) in addition to the CPU 901 or in place of the CPU 901. In this case, the GPU or the FPGA can execute the program according to the present example embodiment.

The CPU 901 carries out various types of computation by deploying the program that constituted by a group of codes according to the present example embodiment stored in the storage device 903 to the main memory 902, and executing the deployed each codes in a predetermined order. The main memory 902 is typically a volatile storage device, such as a DRAM (Dynamic Random Access Memory).

Also, the program according to the present example embodiment is provided in a state where it is stored in a computer readable recording medium 910. Note that the program according to the present example embodiment may also be distributed over the Internet connected via the communication interface 907.

Furthermore, specific examples of the storage device 903 include a hard disk drive, and also a semiconductor storage device, such as a flash memory. The input interface 904 mediates data transmission between the CPU 901 and an input device 908, such as a keyboard and a mouse. The display controller 905 is connected to a display device 909, and controls displays on the display device 909.

The data reader/writer 906 mediates data transmission between the CPU 901 and the recording medium 910, and executes readout of the program from the recording medium 910, as well as writing of the result of processing in the computer 900 to the recording medium 910. The communication interface 907 mediates data transmission between the CPU 901 and another computer.

Also, specific examples of the recording medium 910 include: a general-purpose semiconductor storage device, such as CF (Compact Flash®) and SD (Secure Digital): a magnetic recording medium, such as Flexible Disk; and an optical recording medium, such as CD-ROM (Compact Disk Read Only Memory).

Note that the guidance assistance apparatus 100 according to the present example embodiment can also be realized by using items of hardware corresponding to respective components, rather than by using the computer with the program installed therein. Furthermore, a part of the guidance assistance apparatus 100 may be realized by the program, and the remaining part of the guidance assistance apparatus 100 may be realized by hardware.

A part or all of the aforementioned example embodiment can be described as, but is not limited to, the following (Supplementary Note 1) to (Supplementary Note 19).

### (Supplementary Note 1)

A guidance assistance apparatus comprising:

an acquisition unit acquires biological information of a student measured using a wearable terminal worn by the student:

an estimation unit estimates a feeling of the student based on the acquired biological information; and

a presentation unit presents condition information including the feeling of the student, together with extracurricular activity information on the student's extracurricular activity, on a user terminal used by a teacher.

### (Supplementary Note 2)

The guidance assistance apparatus according to Supplementary Note 1, wherein the presentation unit divides the condition information into condition information of schoolwork and condition information of the extracurricular activity, based on a time of measurement of the biological information.

### (Supplementary Note 3)

The guidance assistance apparatus according to Supplementary Note 1 or 2, wherein the presentation unit executes:

for each of a plurality of students, associating a student image object representing the student with an extracurricular activity object representing the student's extracurricular activity, and presenting the same in a list on the user terminal;

presenting the student image object in different colors according to a feeling toward the schoolwork; and

presenting the extracurricular activity object in different colors according to a feeling toward the extracurricular activity.

### (Supplementary Note 4)

The guidance assistance apparatus according to Supplementary Note 3, wherein when the student image object of one student is selected from the list, the presentation unit presents detailed information of the selected student on the user terminal in a form of a pop-up screen.

### (Supplementary Note 5)

The guidance assistance apparatus according to Supplementary Note 4, wherein the presentation unit further presents, on the pop-up screen, a result of a questionnaire about the student's degree of understanding of classes.

### (Supplementary Note 6)

The guidance assistance apparatus according to any one of Supplementary Notes 1 to 5, wherein the estimation unit estimates, as the feeling, at least one of arousal, valence, and emotion.

### (Supplementary Note 7)

A guidance assistance method comprising:

acquiring biological information of a student using a wearable terminal worn by the student:

estimating a feeling of the student based on the acquired biological information; and

presenting condition information including the feeling of the student to a user terminal used by a teacher, together with extracurricular activity information on the student's extracurricular activity.

### (Supplementary Note 8)

The guidance assistance method according to Supplementary Note 7, wherein the condition information is divided into condition information of schoolwork and condition information of the extracurricular activity, based on a time of measurement of the biological information.

### (Supplementary Note 9)

The guidance assistance method according to Supplementary Note 7 or 8, wherein:

for each of a plurality of students, associating a student image object representing the student with an extracurricular activity object representing the student's extracurricular activity, and presenting the same in a list on the user terminal;

presenting the student image object in different colors according to a feeling toward the schoolwork; and

presenting the extracurricular activity object in different colors according to a feeling toward the extracurricular activity.

### (Supplementary Note 10)

The guidance assistance method according to Supplementary Note 9, wherein when the student image object of one student is selected from the list, presenting detailed information of the selected student on the user terminal in a form of a pop-up screen.

### (Supplementary Note 11)

The guidance assistance method according to Supplementary Note 10, wherein further presenting on the pop-up screen, a result of a questionnaire about the student's degree of understanding of classes.

### (Supplementary Note 12)

The guidance assistance method according to any one of Supplementary Notes 7 to 11, wherein estimating, as the feeling, at least one of arousal, valence, and emotion.

### (Supplementary Note 13)

A computer readable recording medium that includes a program recorded thereon, the program including instructions that causes a computer to function as a guidance assistance apparatus, wherein

the guidance assistance apparatus includes:

an acquisition unit acquires biological information of a student measured using a wearable terminal worn by the student:

an estimation unit estimates a feeling of the student based on the acquired biological information; and

a presentation unit presents condition information including the feeling of the student, together with extracurricular activity information on the student's extracurricular activity, on a user terminal used by a teacher.

### (Supplementary Note 14)

The computer readable recording medium according to Supplementary Note 13, that includes a program recorded thereon, the program including instructions that causes a computer to function as a guidance assistance apparatus, wherein

the presentation unit divides the condition information into condition information of schoolwork and condition information of the extracurricular activity, based on a time of measurement of the biological information.

### (Supplementary Note 15)

The computer readable recording medium according to Supplementary Note 13 or 14, that includes a program recorded thereon, the program including instructions that causes a computer to function as a guidance assistance apparatus, wherein the presentation unit executes:

for each of a plurality of students, associating a student image object representing the student with an extracurricular activity object representing the student's extracurricular activity, and presenting the same in a list on the user terminal:

presenting the student image object in different colors according to a feeling toward the schoolwork; and

presenting the extracurricular activity object in different colors according to a feeling toward the extracurricular activity.

### (Supplementary Note 16)

The computer readable recording medium according to Supplementary Note 15, that includes a program recorded thereon, the program including instructions that causes a computer to function as a guidance assistance apparatus, wherein

when the student image object of one student is selected from the list, the presentation unit presents detailed information of the selected student on the user terminal in a form of a pop-up screen.

### (Supplementary Note 17)

The computer readable recording medium according to Supplementary Note 16, that includes a program recorded thereon, the program including instructions that causes a computer to function as a guidance assistance apparatus, wherein

the presentation unit further presents, on the pop-up screen, a result of a questionnaire about the student's degree of understanding of classes.

### (Supplementary Note 18)

The computer readable recording medium according to any one of Supplementary Notes 13 to 17, that includes a program recorded thereon, the program including instructions that causes a computer to function as a guidance assistance apparatus, wherein

the estimation unit estimates, as the feeling, at least one of arousal, valence, and emotion.

### (Supplementary Note 19)

A guidance assistance system including:

a wearable terminal for acquiring biological information of a student worn the wearable terminal:

a user terminal used by a teacher; and

a guidance assistance apparatus for presenting condition information of the student on the user terminal: wherein

the guidance assistance apparatus comprising:

an acquisition unit acquires biological information of a student measured using the wearable terminal,

an estimation unit estimates a feeling of the student based on the acquired biological information, and

a presentation unit presents condition information including the feeling of the student, together with extracurricular activity information on the student's extracurricular activity, on the user terminal.

Although the invention of the present application has been described above with reference to an example embodiment, the invention is not limited to the example embodiment described above. Various modifications apparent to those skilled in the art can be made to the configurations and details of the invention within the scope of the invention.

### INDUSTRIAL APPLICABILITY

As described above, according to the present disclosure, it is possible to assist teachers in providing guidance to students. The present disclosure is useful in the field of educational assistance.

### LIST OF REFERENCE SIGNS

1 Guidance assistance system
10A User-side apparatus
10B Server-side apparatus
100 Guidance assistance apparatus
110 Acquisition unit
120 Estimation unit
130 Presentation unit
200 Student information DB
221 Class data
223 Student data
225 Student input data
300 Wearable terminal
400 User terminal
500 List screen
510 Pop-up screen
700 (student's) Terminal
800 Questionnaire screen
900 Computer
901 CPU
902 Main memory
903 Storage device
904 Input interface
905 Display controller
906 Data reader/writer
907 Communication interface
908 Input device
909 Display device
910 Recording medium
911 Bus

What is claimed is:

1. A guidance assistance apparatus comprising:
an acquisition unit configured to acquire biological information of a student measured using a wearable terminal worn by the student;
an estimation unit configured to estimate a feeling of the student based on the acquired biological information; and
a presentation unit configured to present condition information including the feeling of the student, together with extracurricular activity information on the student's extracurricular activity, on a user terminal used by a teacher.

2. The guidance assistance apparatus according to claim 1, wherein the presentation unit divides the condition information into condition information of schoolwork and condition information of the extracurricular activity, based on a time of measurement of the biological information.

3. The guidance assistance apparatus according to claim 1, wherein the presentation unit executes:
for each of a plurality of students, associating a student image object representing the student with an extracurricular activity object representing the student's extracurricular activity, and presenting the same in a list on the user terminal;
presenting the student image object in different colors according to a feeling toward the schoolwork; and
presenting the extracurricular activity object in different colors according to a feeling toward the extracurricular activity.

4. The guidance assistance apparatus according to claim 3, wherein when the student image object of one student is selected from the list, the presentation unit presents detailed information of the selected student on the user terminal in a form of a pop-up screen.

5. The guidance assistance apparatus according to claim 4, wherein the presentation unit further presents, on the pop-up screen, a result of a questionnaire about the student's degree of understanding of classes.

6. The guidance assistance apparatus according to claim 1, wherein the estimation unit estimates, as the feeling, at least one of arousal, valence, and emotion.

7. A guidance assistance method comprising:
acquiring biological information of a student using a wearable terminal worn by the student;
estimating a feeling of the student based on the acquired biological information; and
presenting condition information including the feeling of the student to a user terminal used by a teacher, together with extracurricular activity information on the student's extracurricular activity.

8. A non-transitory computer readable recording medium that includes a program recorded thereon, the program including instructions that causes a computer to function as a guidance assistance apparatus, wherein
the guidance assistance apparatus includes:
an acquisition unit configured to acquire biological information of a student measured using a wearable terminal worn by the student;
an estimation unit configured to estimate a feeling of the student based on the acquired biological information; and
a presentation unit configured to present condition information including the feeling of the student, together with extracurricular activity information on the student's extracurricular activity, on a user terminal used by a teacher.

9. The guidance assistance method according to claim 7, wherein the condition information is divided into condition information of schoolwork and condition information of the extracurricular activity, based on a time of measurement of the biological information.

10. The guidance assistance method according to claim 7, wherein:
for each of a plurality of students, associating a student image object representing the student with an extracurricular activity object representing the student's extracurricular activity, and presenting the same in a list on the user terminal;
presenting the student image object in different colors according to a feeling toward the schoolwork; and
presenting the extracurricular activity object in different colors according to a feeling toward the extracurricular activity.

11. The guidance assistance method according to claim 10, wherein when the student image object of one student is selected from the list, presenting detailed information of the selected student on the user terminal in a form of a pop-up screen.

12. The guidance assistance method according to claim 11, wherein further presenting on the pop-up screen, a result of a questionnaire about the student's degree of understanding of classes.

13. The guidance assistance method according to claim 7, wherein estimating, as the feeling, at least one of arousal, valence, and emotion.

14. The non-transitory computer readable recording medium according to claim 8, that includes a program recorded thereon, the program including instructions that causes a computer to function as a guidance assistance apparatus, wherein
the presentation unit divides the condition information into condition information of schoolwork and condition information of the extracurricular activity, based on a time of measurement of the biological information.

15. The non-transitory computer readable recording medium according to claim 8, that includes a program recorded thereon, the program including instructions that causes a computer to function as a guidance assistance apparatus, wherein the presentation unit executes:
for each of a plurality of students, associating a student image object representing the student with an extracurricular activity object representing the student's extracurricular activity, and presenting the same in a list on the user terminal;
presenting the student image object in different colors according to a feeling toward the schoolwork; and
presenting the extracurricular activity object in different colors according to a feeling toward the extracurricular activity.

16. The non-transitory computer readable recording medium according to claim 15, that includes a program recorded thereon, the program including instructions that causes a computer to function as a guidance assistance apparatus, wherein
when the student image object of one student is selected from the list, the presentation unit presents detailed information of the selected student on the user terminal in a form of a pop-up screen.

17. The non-transitory computer readable recording medium according to claim 16, that includes a program recorded thereon, the program including instructions that causes a computer to function as a guidance assistance apparatus, wherein
the presentation unit further presents, on the pop-up screen, a result of a questionnaire about the student's degree of understanding of classes.

18. The non-transitory computer readable recording medium according to claim 8, that includes a program recorded thereon, the program including instructions that causes a computer to function as a guidance assistance apparatus, wherein
the estimation unit estimates, as the feeling, at least one of arousal, valence, and emotion.
